(19) United States
(12) Patent Application Publication
(10) Pub. No.: US 2011/0009326 A1
(43) Pub. Date: Jan. 13, 2011

(54) METHODS OR USE OF LAMIN B1 NUCLEAR ANTIGEN, FRAGMENTS AND COMPOSITIONS THEREOF, FOR INHIBITING OR REDUCING A THROMBOTIC EVENT

(76) Inventors: Yves Raymond, Longueuil (CA); Jean-Luc Senecal, Outremont (CA); Marie-Soleil Christin-Piche, Ste-Anne-De-Bellevue (CA); Nathalie Brassard, St-Jean-Sur-Richelieu (CA)

Correspondence Address: GOUDREAU GAGE DUBUC, 2000 MCGILL COLLEGE, SUITE 2200, MONTREAL, QC, H3A 3H3, CA

(21) Appl. No.: 11/572,170

(22) PCT Filed: Jul. 15, 2005
(86) PCT No.: PCT/CA2005/001108

§ 371 Date: Jul. 31, 2008

Related U.S. Application Data
(60) Provisional application No. 60/588,327, filed on Jul. 16, 2004.

(51) Int. Cl. A61K 38/36; A61P 7/02

(52) U.S. Cl. 514/14.9

(57) ABSTRACT

A method for preventing a thrombotic event in a patient susceptible to such an event, which comprises the step of administering an effective amount of a lamin 131 nuclear (LB1) antigen to said patient is provided along with an anti-thrombotic composition which comprises an effective amount of a lamin 131 nuclear (LB1) antigen and a pharmaceutically acceptable carrier.

### CROSS REFERENCE TO RELATED APPLICATION

This application claims priority on U.S. provisional application No. 60/588,327, filed on Jul. 16, 2004. All documents above are herein incorporated by reference.

### FIELD OF THE INVENTION

The present invention relates to methods of use of the nuclear autoantigen lamin B1 and fragments thereof and composition thereof.

### BACKGROUND OF THE INVENTION

Thrombosis is the inappropriate or pathological formation of an obstructive clot from the constituents of blood, a thrombus, within a blood vessel or organ. Depending on the location of the clot, the resultant loss of circulation can lead to a stroke (cerebral thrombosis) or a heart attack (coronary thrombosis). Individuals affected by certain diseases and conditions are susceptible to thrombosis.

Systemic lupus erythematosus (SLE) is one such disease. It is an autoimmune disease characterized by circulating autoantibodies, which are associated with numerous clinical manifestations (1-3). One family of autoantibodies, called antiphospholipid antibodies (aPL), are known to contribute to the pathogenesis of the antiphospholipid syndrome (APS) often observed in SLE patients. APS is characterized by the occurrence of arterial and venous thrombosis or recurrent pregnancy loss in the presence of aPL (1-3). The presence of lupus anticoagulant (LAC), a subset of aPLs, in these patients, is a strong predictor for thrombosis (4) since 50% of LAC positive patients have been found to eventually develop thrombotic episodes (5).

Recent observations have shown that LAC positive patients who also have high titers of autoantibodies directed against the nuclear autoantigen lamin B1 (LB1) have a lower frequency (22.7%) of thrombotic manifestations than LAC positive and anti-LB1 negative patients (50%) (6, 7). It was initially postulated that the anti-LB1 antibodies could confer protection against the procoagulant effect of LAC (6). Studies with apoptotic blebs (6), endothelial cells (Dieudé, personal communication), coagulation factors and platelets (unpublished data) demonstrated that anti-LB1 antibodies on their own did not seem to have any effect on the main pathways or important cells involved in coagulation.

The nuclear lamina is a protein meshwork that lines the inner nuclear membrane and plays a critical role in many fundamental processes including spatial organization of chromatin, DNA replication, and gene transcription (13). The principal protein components of the lamina are lamins, which are members of intermediate filament protein family. Like other intermediate filament proteins,. lamins possess a highly conserved central α-rod domain for polymerization (13). LB1 is one of the components of the nuclear lamina. During apoptosis, LB1 is cleaved by caspase-6 into 35 kDa and 49 kDa fragments, which are then packaged inside apoptotic blebs between the aspartic acid residue at position 231 and the serine residue at position 232 (6). Release of this autoantigen into the extracellular medium is normally prevented by swift removal of apoptotic debris. However, in many autoimmune diseases, some autoantigens are released in the extracellular environment due to defects in the apoptotic debris clearance mechanisms (14-16).

There remains a need to better characterize the role of LB1 in thrombosis.

The present invention seeks to meet these needs and other needs.

The present description refers to a number of documents, the content of which is herein incorporated by reference in their entirety.

### SUMMARY OF THE INVENTION

The Applicant is the first to have identified a role for LB1 in platelet function. The present invention thus relates to the binding properties of the autoantigen LB1 on platelets and the effect of such binding on the activation and aggregation of these cells. The Applicant is the first to demonstrate that LB1 impairs the externalization of P-selectin (also called herein CD62) and CD63 on platelets stimulated with thrombin. Furthermore, the Applicant is the first to establish that LB1 decreases the activation of the GPIIb/IIIa complex at the platelet surface and diminishes platelet aggregation following stimulation with thrombin, collagen, phorbol myristate acetate (PMA), and thrombin activating peptides (TRAP) 1 and 4. The Applicant is also the first to show that LB1 binds directly to targets located within platelets and that its entry appears to occur exclusively during platelet activation. The present invention is the first to demonstrate the capability of an autoantigen to impair platelet activation and aggregation, and thus, identifies a role for this molecule in antithrombotic therapies and prevention. It is to be noted that in a thrombotic event, the population of platelets comprises cells at all stages of activation including cells at a stage within the action window of LB1.

As used herein, the term “pharmaceutically acceptable carrier” refers to solutions, suspension, or tablets prepared with commonly used excipients such as those described in Modern Pharmaceutics, 4th edition. Banker G S and Rhodes C T (eds) Marcel Dekker, NY, 2002. It also refers to any suitable form of immediate, controlled, delayed, and slow release formulations or devices (liposomes, implants, stents . . . ) and any suitable parenteral vehicles. The release kinetics may be constant or variable e.g. rapid at the beginning and slower with time depending on a decreasing concentration gradient.

As used herein, the term “Lamin B1 antigen” or “LB1 antigen” refers to the full length LB1 protein or to a functional C-terminal fragment thereof. The “full length LB1 protein” refers herein to any known human variant of the LB1 protein prior to it being subjected to the caspase-6 catalytic action including the LB1 presented in FIG. 9. It also includes any mammalian species variant of this protein. The term “functional C-terminal fragment” includes the 49 kDa LB1 fragment derived from the catalytic action of the caspase-6 and the recombinant 49 kDa fragment disclosed herein, along with any smaller fragment thereof that retains its ability to prevent or reduce thrombotic events.

As used herein, the term “effective amount” of a LB1 composition of the present invention refers to an amount that is effective for inhibiting or preventing thrombus formation. Without being so limited, the effective amount of LB1 administered in situ to patients in need thereof may be in an amount from about 0.001 mg up to about 50 mg per day or in one single bolus dose, more specifically, from about 0.01 mg to 10 mg, even more specifically from about 0.1 to 5 mg. The term “administration in situ” refers herein to an administration that is in close proximity (i.e. on or within the blood vessel itself or within the blood vessel wall) to the location within a blood vessel lumen where there is a risk of thrombus formation. There are risks of thrombus formation in locations for instances where a thrombus/clot or an atherosclerosis plaque occurred, where there are risks of stenosis or restenosis, at locations of vascular injuries including those caused by angioplasty including percutaneous transluminal coronary angioplasty (PTCA). Such locations also include any putative thrombus formation sites generated by surgery of any sort. In situ administration may be performed for instance with the help of a catheter, a stent, a tablet or implant placed within a vessel wall with provides controlled release of the LB1 antigen, etc.

As used herein, the term “repetitive basis” refers to the more or less continuous administration of LB1 in order to inhibit or prevent thrombosis, as opposed to a single administration. The repetitive basis may take the form of a daily administration of LB1 or of a continuous release from a slow release system, or a combination of both i.e. a bolus and a slow release to keep the concentration of LB1 at a substantially constant active level at the site of thrombosis.

As used herein, the term “thrombotic event” refers to the steps of the formation of a thrombus and to its associated processes e.g. externalization of platelet P-selectin and CD63, GPIIb/IIIa complex activation and platelet aggregation.

As used herein, the term “platelet activation” refers to externalization of P-selectin and CD63, and GPIIb/IIIa complex activation.

In accordance with the present invention, there is therefore provided a method for preventing a thrombotic event in a patient susceptible to such an event, which comprises the step of administering an effective amount of a lamin B1 nuclear (LB1) antigen to said patient.

In accordance with another aspect of the present invention, there is provided a method for reducing a thrombotic event in a patient in need for such a treatment, which comprises the step of administering an effective amount of a lamin B1 nuclear (LB1) antigen to said patient.

In specific embodiments of these methods, the LB1 antigen is a full length LB1. In other specific embodiments, the full length LB1 is human. In other specific embodiments, the LB1 antigen is a 49 kDa human LB1 C-terminal fragment. In other embodiments, the effective amount of a LB1 antigen is administered in situ. According to specific embodiments, the thrombotic event comprises platelet P-selectin externalization and/or platelet CD63 externalization and/or platelet GPIIb/IIIa complex activation and/or platelet aggregation. In other specific embodiments, the effective amount of LB1 antigen is administered to said patient prior to platelet activation. In other specific embodiments, the effective amount of a LB1 antigen is administered to said patient during platelet activation.

According to another aspect of the present invention, there is provided a use of a lamin B1 nuclear (LB1) antigen for the prevention of a thrombotic event.

According to a further aspect of the present invention, there is provided a use of a lamin B1 nuclear (LB1) antigen for the preparation of a medicament for the prevention of a thrombotic event.

According to a further aspect of the present invention, there is provided a use of a lamin B1 nuclear (LB1) antigen for the reduction of a thrombotic event.

According to a further aspect of the present invention, there is provided a use of a lamin B1 nuclear (LB1) antigen in the preparation of a medicament for the reduction of a thrombotic event.

In specific embodiments of these uses, the LB1 antigen is a full length LB1. In other specific embodiments, the full length LB1 is human. In other specific embodiments, the LB1 antigen is a 49 kDa human LB1 C-terminal fragment. In other embodiments, the effective amount of a LB1 antigen is administered in situ. According to specific embodiments, the thrombotic event comprises platelet P-selectin externalization and/or platelet CD63 externalization and/or platelet GPIIb/IIIa complex activation and/or platelet aggregation. In other specific embodiments, the effective amount of LB1 antigen is administered to said patient prior to platelet activation. In other specific embodiments, the effective amount of a LB1 antigen is administered to said patient during platelet activation.

There is also provided an anti-thrombotic composition which comprises an effective amount of a lamin B1 nuclear (LB1) antigen and a pharmaceutically acceptable carrier.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

FIG. 1 shows the results of SDS-PAGE following purification of LB1. Lane 1 shows molecular weight standards in kDa; lane 2, crude bacterial lysate extract; lane 3, flow-through fraction from the Ni-affinity column; and lane 4, 1.5 mg of LB1. Bands were stained with Coomassie blue;

FIG. 2 graphically illustrates the effect of LB1 on platelet degranulation. Panel A shows flow cytometry histograms of P-selectin expression. Panel B shows dose-response inhibition curves of CD62 externalization. Panel C shows a bar graph showing the expression of platelet CD63 following treatment with 200 ng of LB1 or control proteins/10⁶ platelets. Percentages of P-selectin positive cells are the mean and SEM representative of three independent experiments done in duplicates. Percentages of CD63 positive cells are the mean and SEM representative of three independent experiments done in duplicate;

FIG. 3 graphically illustrates the effect of full length human lamin B1 (LB1), and of its N-terminal (35 kDa) and C-terminal (49 kDa) fragments on platelet degranulation through a dose-response curve of CD62 surface expression on thrombin-activated platelets;

FIG. 4 illustrates through a bar graph the effect of LB1 on GPIIb/IIIa complex activation. Percentages of PAC-1 positive cells are the mean and SEM representative of three independent experiments done in duplicate;

FIG. 5 graphically illustrates the effect of LB1 on platelet aggregation stimulated with either thrombin (Panel A), collagen (Panel B), PMA (Panel C), TRAP1 (Panel D) or TRAP4 (Panel E); Platelet aggregation tracings are representative of 4 independent experiments.

FIG. 6 graphically illustrates OD values of LB1 binding to permeabilized platelets with increasing LB1 concentration. Values are the mean and SEM representative of three independent experiments done in triplicate;

FIG. 7 illustrates localization of LB1 binding sites by double indirect immunofluorescence and confocal microscopy. Green stains denote LB1 presence while red staining denote cell membrane. Panel A shows anti-LB1 IgG (green) and mouse anti-CD61 antibody (red) as a cell surface marker; Panel B shows horizontal optical sections of platelets stained with anti-LB1 and anti-CD61. DIC represents differential imaging contrast; Panel C shows unactivated platelets pretreated with LB1 and incubated with an anti-LB1 IgG and a mouse anti-CD61 antibody; and Panel D shows activated platelets incubated with an anti-LB1 IgG (green) and a mouse anti-CD62/P-selectin antibody (red) as an activation marker. The images shown are representative of 3 independent experiments. Bars=5 μm and the arrow in FIG. 6A points to a platelet with blunt filopodia rotated around the cell periphery;

FIG. 8 illustrates through a bar graph the LB1 activity as a function of platelet activation state. Mean fluorescence intensities (MFI) are the means and SEM representative of three independent experiments done in duplicate; and

FIG. 9 shows the amino acid sequence (SEQ ID NO: 1) of the human LB1.

### DESCRIPTION OF ILLUSTRATIVE EMBODIMENTS

### Purification of LB1 and NH₂-LB1

### LB1

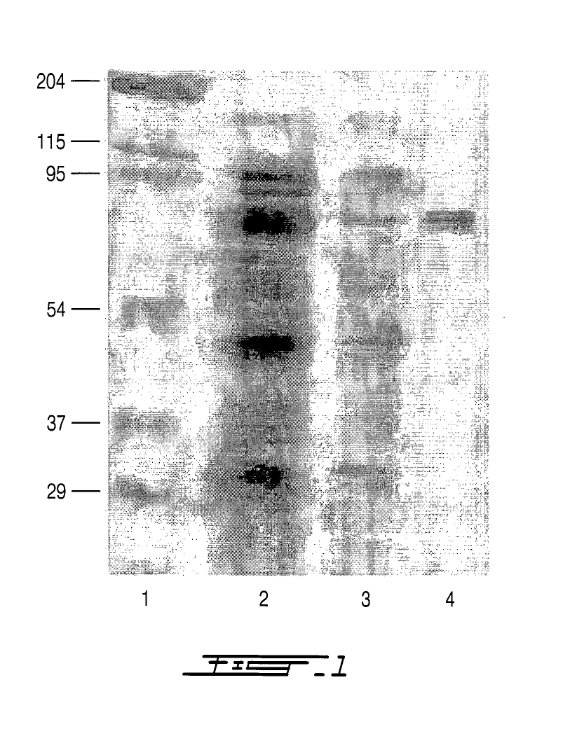

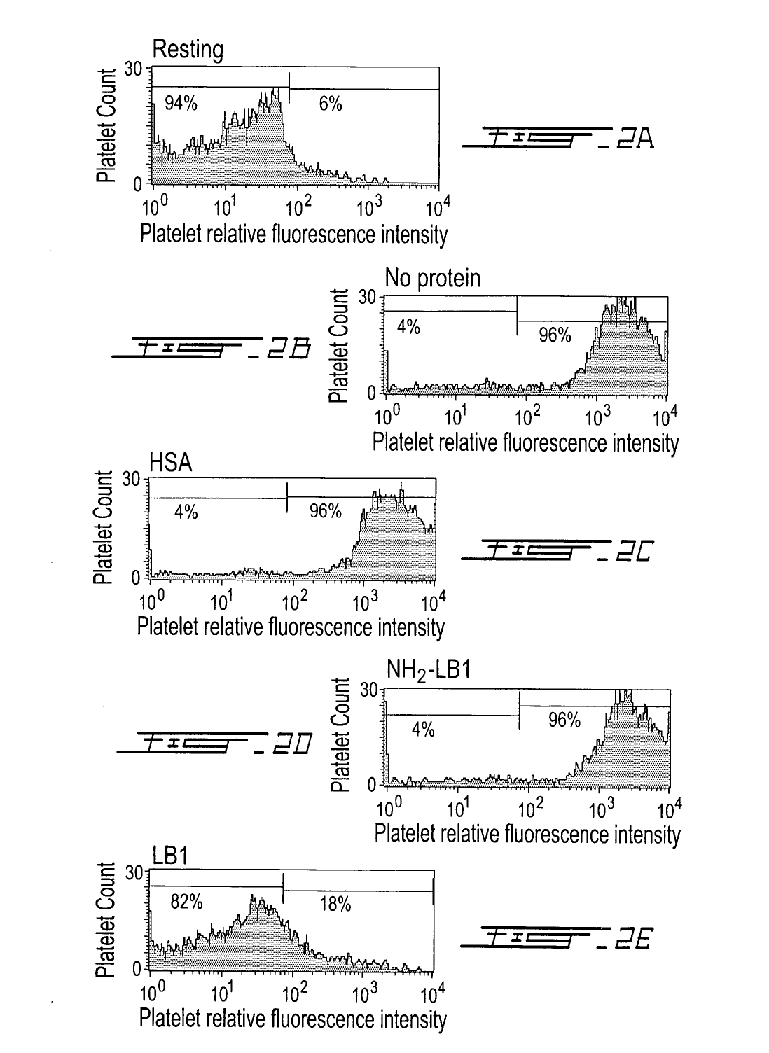

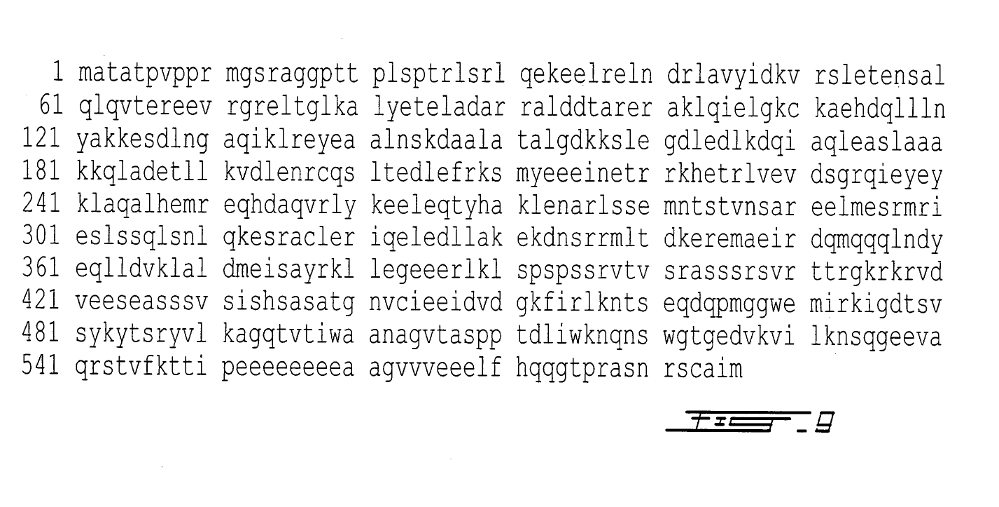

The expression vector coding for the human LB1 gene has been previously described (6). Briefly, BL21(DE3) E. coli (Stratagene, La Jolla, Calif.) cells bearing the plasmid pET19b-LB1 that codes for full length LB1 (Accession AAC37575, GI 576840 and FIG. 9) were grown overnight at 25° C. in LB media supplemented with carbenicillin [100 μg/ml]. The overnight culture of E. coli BL21 (DE3) was diluted 1:25 in fresh medium and incubated at 32° C. until the OD₆₀₀ reached 0.6. Cells were then induced at 32° C. with 0.7 mM isopropyl-b-D-thiogalactopyranoside (Sigma, St-Louis, Mo.) for 2 h at 32° C. LB1 was purified with Ni-NTA resin (Novagen, Madison, Wis.). One g of bacterial pellet was resuspended in 20 mL of extractor buffer (BD Pharmingen, Mississauga, ON), 40 units of DNAse (Sigma) and 20 mg lysozyme for 10 minutes at room temperature (RT) and then sonicated. The cell lysate was centrifuged at 20 000×g for 20 min at 4° C. The supernatant was recovered, poured onto 10 ml of Ni-NTA resin and incubated for 20 min at RT. The resin bed was washed with 0.5 M NaCl, 0.05 M sodium phosphate buffer and 25 mM imidazole, pH 8.0 (B1 buffer) and 0.5 M NaCl, 0.05 M sodium phosphate buffer and 50 mM imidazole, pH 8.0 (B2 buffer). Finally, the protein was eluted with 0.5 M NaCl, 0.05 M sodium phosphate buffer and 250 mM imidazole, pH 8.0. LB1 was concentrated in Centricon concentrators (Millipore, Billerica, Mass.) up to approximately 0.25 μg/μl and the buffer was exchanged for Tyrode's buffer. The purity of the sample was assessed by SDS-PAGE (FIG. 1).

### LB1-COOH

The truncated C-terminal fragment (LB1-COOH, 49 kDa) of the human LB1 gene was generated by insertion of a start codon (ATG) in front of amino acid position 233 (glycine) by a gene synthesizer (Operon) yield the fragment Gly₂₃₃-Met₅₈₆. Subcloning into the pET19B expression vector added the deca-histidine tag at the N-terminus of LB1-COOH together with an extra 24 amino-acid sequence from the vector. Transformation was into E. coli BL21 (Al) for expression. Production was induced by the addition of 0.2% arabinose and incubation at 30° C. for 2 h. Purification was carried out as described for LB1 except for the following modifications. Bacterial pellets from 25 ml of culture were resuspended in 0.05 M sodium phosphate, 0.5 M sodium chloride, 10 mM imidazole buffer, pH 8.0 and treated with lysozyme and sonication. Chromatography was performed on Ni-NTA resin with wash buffers containing 25 and then 50 mM imidazole, and elution buffers containing 250 and then 500 mM imidazole. Eluates were pooled and concentrated as was LB1.

### NH₂-LB1

The truncated N-terminal fragment (NH₂-LB1, 35 kDa) of the human LB1 gene was generated by insertion of a stop codon (TAA) at amino acid position 232, i.e. following the aspartic acid residue at position 231, by a gene synthesizer (Operon Technologies Alamed, Calif.). NH₂-LB1 was inserted into pET19b expression vector (Novagen) and transformed into E. coli BL21 (DE3) for expression. Production and purification of the protein was carried out as described for pET19b-LB1 except for the following modifications. A bacterial pellet of 0.2 g was resuspended in 4 mL of extractor buffer (BD pharmingen) supplemented with 40 units of DNAse and 0.4 mg lysozyme. Purification was performed with 2 ml of Ni-NTA resin. Prior to elution, the resin bed was washed with B1 and B2 buffers as well as 0.5 M NaCl, 0.05 M sodium phosphate buffer and 100 mM imidazole, pH 8.0.

### Preparation of Human Platelets

### Flow Cytometry, Immunofluorescence and ELISA

Venous blood was withdrawn from healthy human volunteers free from any medication that interfere with platelet functions for at least 10 days and anticoagulated with sodium citrate. Concentrated platelet-rich plasma (PRP) was obtained by centrifuging the blood at 150×g for 15 minutes at 25° C. Five mM EDTA and 5.6 μM prostaglandin (PGE) (Sigma) were added to the PRP. Platelets were then pelleted at 1000×g for 10 min and resuspended in Tyrode's buffer pH 7.4, free of Ca²⁺. Platelets were counted with an automated blood cell counter and the concentration was adjusted at 500×10⁶ platelets/mL.

### Aggregometry

Venous blood was withdrawn as described above and anticoagulated with acid-citrate dextrose. PRP was obtained by centrifuging the blood at 500×g for 15 minutes at 25° C. Platelets were then pelleted at 800×g for 10 minutes and resuspended in Hank's balanced salt sodium-HEPES buffer with 0.4 mM EDTA (HBSS-EDTA) and 1 mM PGE, pH 6.5. Finally, platelets were centrifuged at 520×g for 8 min and resuspended in HBSS-HEPES buffer pH 7.4 containing 1.3 mM CaCl2 and 0.81 mM MgCl₂. The platelet count was adjusted to 250×10⁶ platelets/mL.

### Flow Cytometry

Resting platelets (25×10⁶ platelets) were incubated with 200 μg/mL goat IgG (Sigma-Aldrich) for 15 min in a polystyrene 96 well plate (Immulon 1HB; Thermolab Systems, Franklin, Mass.) to block non-specific binding sites. For detection of platelet selectin (CD62P), increasing concentrations of LB1, NH₂-LB1 or human serum albumin (HSA; Sigma) were added to each well. For CD63 surface expression and GPIIb/IIIa complex activation experiments, 200 ng of LB1, HSA or NH₂-LB1 was added to 10⁶ platelets per well. Platelets were activated for 15 min with 0.05 units/mL of thrombin (Sigma) and 2 mM CaCl₂. Activated platelets were then incubated with phycoerythrin-conjugated anti-CD62P (1:50, BD Pharmingen), phycoerythrin-conjugated anti-CD63 (1:7, BD Pharmingen) or fluorescein conjugated anti-PAC1 (1:10, BD Pharmingen) for 20 min in the dark. Fluorescence was detected with a FACScan™ and analyzed with CellQuest™ software (BD Biosciences, San Jose, Calif.). The experiment was repeated with 3 different platelet donors.

### Aggregation

Optical platelet aggregation was monitored using a 4-channel platelet aggregation profiler (Chrono-Log, Corporation, Havertown, Pa.). Isolated platelets in HBSS-HEPES buffer were placed in glass cuvettes with 200 ng/10⁶ platelets of LB1, HSA or NH₂-LB1 and incubated for 5 min at 37° C. Samples were placed in the aggregometer with a stirring speed of 1000 rpm and 0.1 units/mL thrombin, 1 μM phorbol myristate acetate (PMA; Chronolog, Havertown, Pa.), 2 μg/mL collagen (Chronolog), 5 μM thrombin activating peptide 1 (TRAP-1; Chronolog) or 125 mM thrombin activating peptide 4 (TRAP-4, Chronolog) was added, and aggregation was monitored for 5 min. The experiment was repeated with 4 different donors.

### Platelet Based Enzyme-Linked Immunosorbent Assay (ELISA)

Polystyrene 96 well plates (Immulon 2HB™) were coated overnight at 4° C. with thrombin (0.05 units/mL) activated human platelets (2.5×10⁶/well) in Tyrode's buffer. Plates were then centrifuged at 220×g for 5 min and washed three times with PBS containing 0.05% Tween-20™ (PBST), plates were centrifuged at 220×g for 5 min. Increasing concentrations of LB1 diluted in Tyrode's buffer were added for 15 min and wells were washed three times with PBST. Wells were blocked with 200 μL of Tyrode's buffer containing 1% BSA (Sigma) and 150 μg/mL goat IgG for 2 h and washed three times with PBST. One hundred μL of mouse anti-CD61 (1:500; BD Pharmingen) or guinea pig anti-LB1 (1:500; Dieudé et al., 2002) diluted in Tyrode-1% BSA were added to each well for 1 h. After washing three times with PBST, plates were incubated for 1 h with horseradish peroxidase conjugated goat anti-mouse (1:5000; Jackson ImmunoResearch, West Grove, Pa.) or goat anti-guinea pig (1:5000; Jackson). Wells were washed three times and peroxidase activity was detected with 8 mg/mL o-phenylenediamine (Sigma) in citrate buffer, pH 6.0, and 0.006% H₂O₂. The reaction was stopped after 10 min with 2M H₂SO₄ and the optical density was read at 490 nm in an MRX Revelation microplate reader™ (Dynex, Chantilly, Va.).

### Immunofluorescence and Confocal Microscopy

Two million platelets (50 μL) were incubated with 200 ng of LB1 in a polystyrene 96 well plate (Immulon 1HB™) for 10 min at 25° C. and then activated with 0.05 U/mL thrombin and 2 mM CaCl₂ for 3 min. Activated platelets were centrifuged at 220×g for 5 min and the supernatant was discarded to remove any unbound LB1. Platelets were resuspended in 100 μL of Tyrode's buffer and placed on glass coverslips covered with 900 μL of Tyrode's buffer. Platelets were centrifuged at 220×g for 5 min, the supernatant was discarded and coverslips were washed 2 times with Tyrode buffer containing 2% BSA (Sigma). Platelets were fixed with 2% paraformaldehyde (Sigma) for 10 min and permeabilized with 0.5% Triton-X-100™ for 10 min. After washing 4 times with Tyrode containing 2% BSA, the coverslips were blocked with 2% BSA and 150 μg/mL goat IgG for 15 min at 25° C. Fixed cells were first incubated with 9 μg/mL mouse anti-LB1 (Zymed, San Francisco, Calif.) and with either rabbit anti-CD61 (1:250; RDI, Flander, N.J.) or rabbit anti-CD62 (2 μg, BD Pharmingen), for 1 h at 25° C. Following 4 washes, bound antibodies were revealed by a 45 min incubation period at 25° C. in the dark with fluorescein-conjugated anti-mouse IgG (1:200, Molecular Probes, Eugen, Oreg.) and rhodamine conjugated anti-rabbit (0.5 mg/mL, Molecular Probes). Coverslips were washed 4 times and mounted with Prolong Gold™ (Molecular Probes) onto microscope slides. Cells were then examined under a 63×oil immersion objective with a Zeiss 510™ (Zeiss, Thornwood, N.Y.) confocal laser microscope.

### Choice of Control Proteins

For flow cytometry, aggregometry and ELISA, human serum albumin (HSA) and NH₂-LB1 were used as control proteins. Since the possibility existed that LB1 was present in the serum of patients, HSA, an abundant serum polypeptide, was chosen as a control. The LB1 is a recombinant polypeptide purified over a nickel-bearing resin. Some contaminants due to the method of purification could be present in the LB1 solution. Since NH₂-LB1, a truncated form of LB1, is a recombinant polypeptide expressed with the same vector, harbouring the same histidine tag and purified in the same way as LB1, it was used as a control.

### Rat Model of Thrombosis

Male Sprague-Dawley rats (weight 350-450 g) were anesthetized with ketamine-xylazine at 50 mg/kg and 5 mg/kg i.m., respectively and maintained with isofurane (1%). Femoral artery and vein were canulated for blood pressure and heart rate monitored during drug administration. Carotid flow was continuously monitored by using an ultrasound flow probe (Transonic) to determine the precise time of occlusion. The left carotid was exposed through a medial ventral longitudinal incision. A Qtip™ was soaked in a FeCl₃ (50% wt/vol) solution for 3 minutes and applied to the ventral surface of the artery, distal to the flow probe, after a stabilization period of 15 min. Complete occlusion was observed in the control group within 60 min, generally between 20-30 min, after the application of the FeCl₃ solution. A residual flow 60 minutes after the application indicated that the thrombus was not completely occlusive and thus that the treatment was anti-thrombotic. This protocol is in accord with the one published earlier (41).

At the end of the experiment, the exposed artery segment was completely excised. The thrombus was then removed from the artery and weighted. A decrease in thrombus weight indicates the ability of the product to reduce thrombosis.

### Example 1

### Effect of LB1 on Granule Secretion

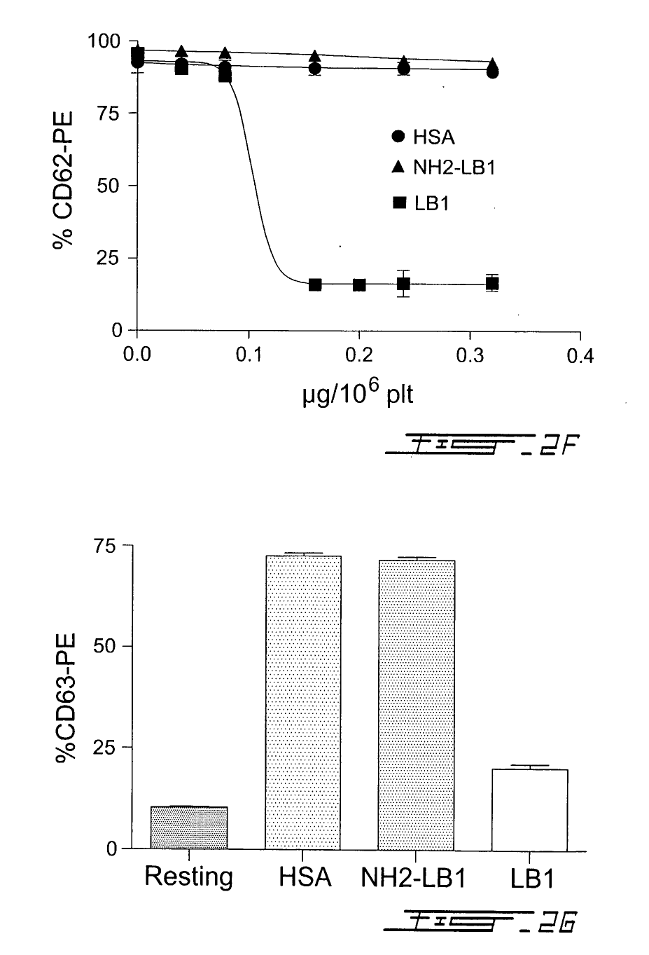

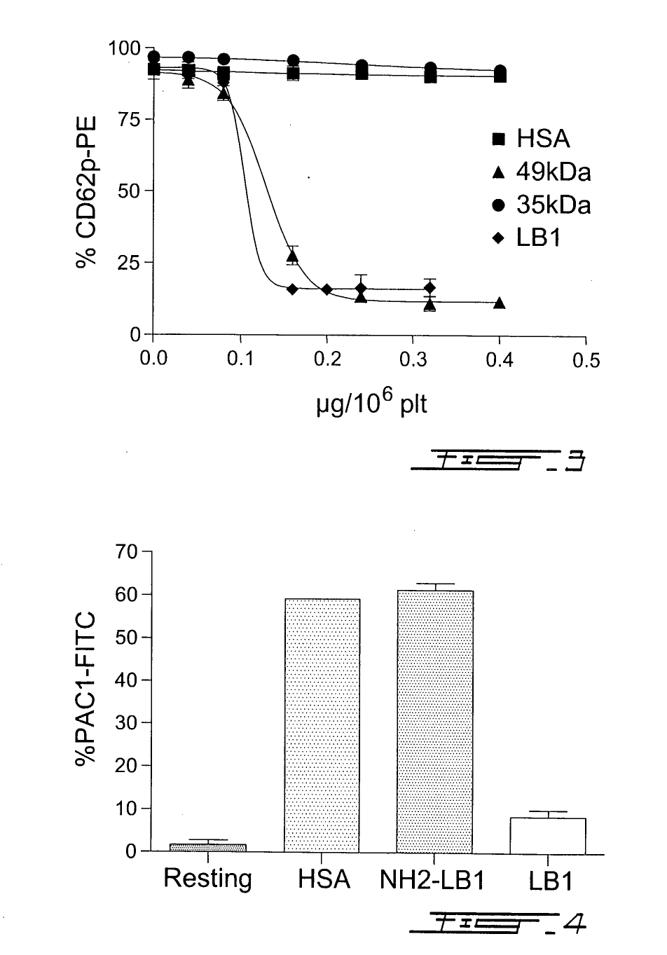

Platelets are secretory cells that release the content of their intracellular granules in response to cellular activation. P-selectin, present in α-granules, and CD63, a lysosomal/dense granule protein, are redistributed to the cell surface of platelets following activation. Because P-selectin/CD62 and CD63 are expressed on degranulated but not resting platelets, these two markers were used to determine the effect of LB1 on platelet activation. Isolated human platelets were treated with varying concentrations of LB1, HSA or NH₂-LB1 before activation with 0.05 U/mL of thrombin. Analysis by flow cytometry as described above thus revealed that full length LB1 diminished the translocation of P-selectin in platelets activated with thrombin (0.05 U/mL) as shown by the shift of the P-selectin fluorescence peak to the left (FIG. 2A), whereas HSA and the truncated NH₂-LB1 did not (FIG. 2A). In the presence of 200 ng of LB1/10⁶ platelets, the percentage of cells displaying surface P-selectin was 15.7±0.9%, whereas percentages of 91.3±1.7% and 95.7±0.5% were measured in the presence of HSA or NH₂-LB1, respectively (FIG. 2B). Incubation of platelets with higher doses of LB1 did not further decrease the externalization of P-selectin. Therefore, the ratio of 200 ng of LB1/10⁶ platelets was used in all flow cytometry experiments.

LB1 had a similar effect on the translocation of the dense/lysosomal surface marker CD63. LB1 decreased externalization of CD63 to the cell surface when compared to HSA or NH₂-LB1 (FIG. 2C). The percentage of CD63 at the surface of thrombin-activated platelets incubated with LB1 was 19.9±0.7%, compared to 73.1±0.6% and 71.5±0.7% with HSA or NH₂-LB1, respectively. Thus, LB1 appears to inhibit both dense granule and lysosome secretion.

### Example 2

### Effect of the 49 kDa Fragment of LB1 on Granule Secretion

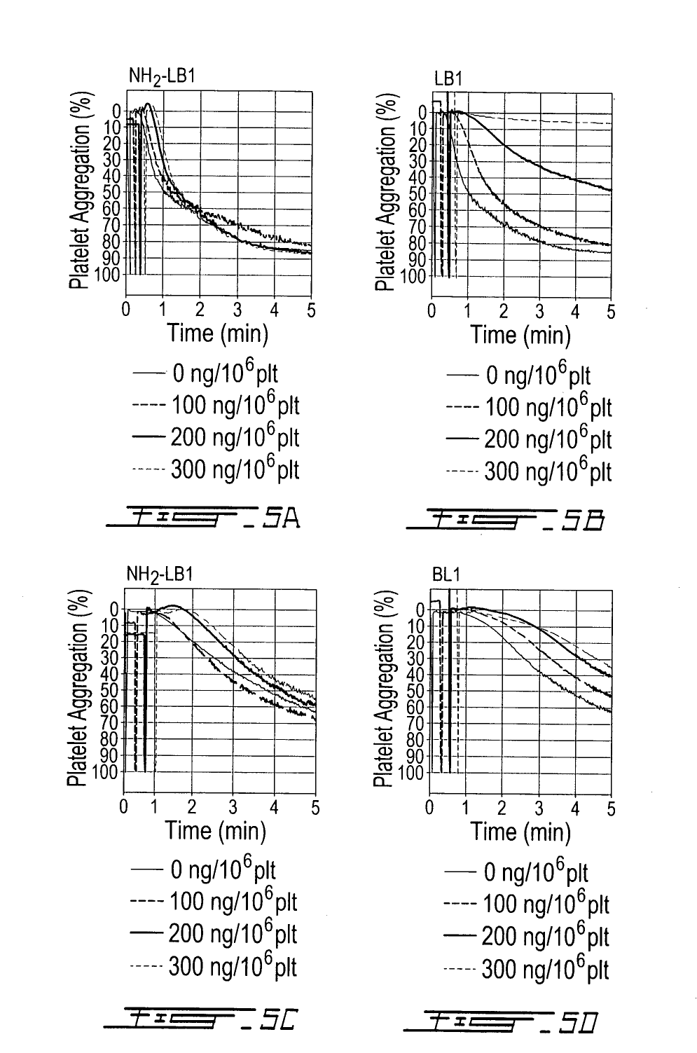

As shown in the accompanying FIG. 3, the 49 kDa truncated LB1-COOH inhibited the externalization of P-selectin by thrombin-activated platelets to the same extent as the full length LB1, albeit on an equivalent weight basis.

This shows that not only LB1 but also a C-terminal fragment of LB1 is effective for the inhibition of platelet activation.

### Example 3

### Effects of LB1 on GPIIb/IIIa Complex Activation

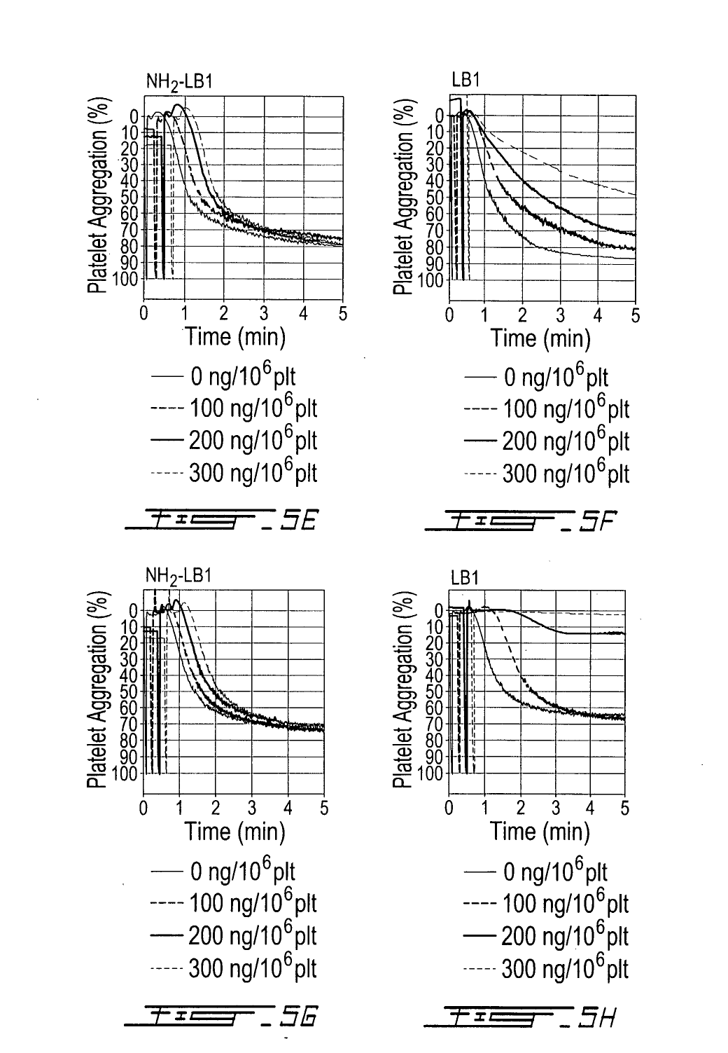

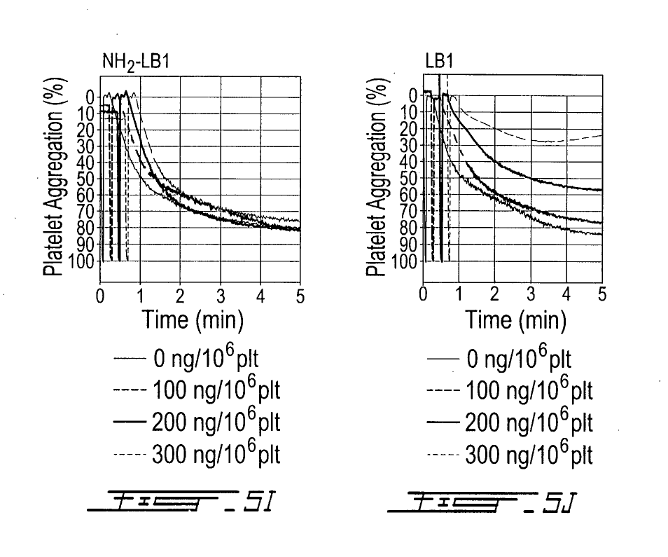

In resting platelets, the aIIbb3 integrin, also called the GPIIb/IIIa complex, maintains a low binding activity for its ligands. Following platelet exposure to soluble agonist, the GPIIb/IIIa complex switches from an inactive to an active state, which increases its ability to bind its ligands, a process that is essential for platelet aggregation. In the presence of LB1, thrombin-activated platelets were unable to present the active conformation of this complex, as measured by platelet activator complex (PAC1) antibody binding (FIG. 4). Analysis by flow cytometry revealed that GPIIb/IIIa complex activation was reduced substantially after incubation with LB1. Isolated human platelets were treated with 200 ng of LB1, HSA or NH₂-LB1/10⁶ platelets before activation with 0.05 U/mL of thrombin. The percentage of active GPIlb/Illa at the platelet surface was only 8.42±1.1% in the presence of LB1 as compared to 59% and 61.02±1.18% in the presence of HSA and NH₂-LB1, respectively.

### Example 4

### Effect of LB1 on Platelet Aggregation

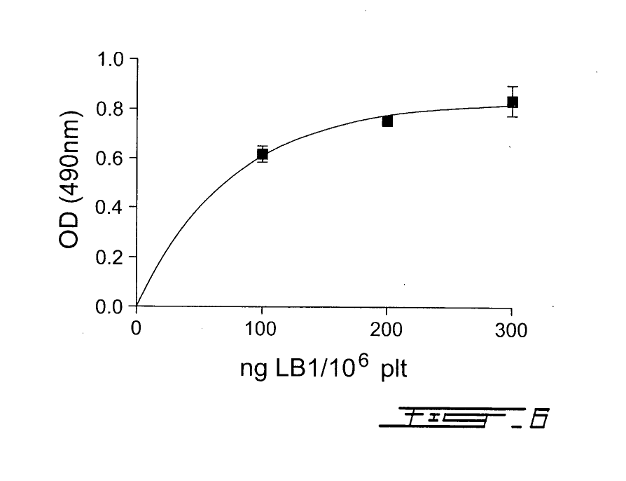

To determine the extent to which LB1 interfered with platelet function, its effect on platelet aggregation was evaluated. In order to determine if LB1 targeted specific pathways of activation, its ability to affect aggregation was measured in the presence of 5 different agonists: thrombin, collagen, phorbol myristate acetate (PMA), thrombin PAR 1 activating peptide (TRAP 1) and thrombin PAR 4 activating peptide (TRAP 4). Isolated human platelets were thus treated with different concentrations of LB1 or NH₂-LB1 before activation with A, 0.1 U/mL of thrombin, B, 2 μg/mL collagen, C, 1 μM PMA, D, 5 μM TRAP-1 or E, 125 μM TRAP-4. Analysis by aggregometry as described above revealed that, as shown in FIG. 5, LB1 was able to retard and decrease the aggregation of platelets in the presence of all agonists tested, compared to NH₂-LB1 and HSA (data not shown). Platelet aggregation was retarded but not diminished in the presence of 100 ng of LB1/10⁶ platelets. However, aggregation of platelets stimulated with all the agonists tested was decreased following treatment with 200 ng of LB1/10⁶ platelets. The aggregation of platelets was diminished by 25% following stimulation with thrombin (FIG. 5A), by 50% after the addition of collagen (FIG. 5B), by 20% after PMA (FIG. 5C) as well as by 25% and 17% following activation with TRAP 1 (FIG. 5D) and TRAP-4 (FIG. 5E), respectively. When LB1 was added at a concentration of 300 ng/10⁶ platelets, neither thrombin nor collagen were able to provoke platelet aggregation. PMA, TRAP-1 and TRAP-4 were still able to aggregate the platelets in the presence of 300 ng of LB1/10⁶ platelets, but the aggregation was reduced when compared to the platelets incubated with 200 ng of LB1/10⁶ platelets. Indeed, the percentage of aggregation with 300ng of LB1/10⁶ cells was 25%, 35% and 60% when the platelets were activated with PMA, TRAP 1 and TRAP 4, respectively. Thus, LB1 appears to affect a pathway of platelet activation that is common to all the agonists tested.

Platelet aggregation is mediated by the binding of fibrinogen or von Willebrand factor to the GPIlb/Illa complex. Activation of this complex is required for aggregation and its blockade prevents thrombus formation (17). LB1 inhibits aggregation induced by thrombin and collagen, and diminishes aggregation stimulated by TRAP 1, TRAP 4 and PMA, an activator of PKC. Since LB1 is able to interfere with platelet aggregation regardless of the agonist used, it must block an important common signalling pathway involved in the activation of platelets. The blockage of GPIIb/IIIa complex activation by LB1 might be at the source of reduced platelet aggregation in the presence of the polypeptide. The inhibition of platelet aggregation and of GPIIb/IIIa complex activation by LB1 is of great interest since it is known that GPIIb/IIIa inhibitors have beneficial effects during percutaneous coronary interventions and acute coronary syndromes (18), as seen for example with the use of Abciximab (19).

Known inhibitors of GPIlb/Illa complex formation are capable of inhibiting aggregation stimulated by different platelet activators, but they have no effect on P-selectin externalization (18, 20). Persistent platelet activation in vivo can contribute to thrombus formation through the generation of platelet-leukocyte complexes, an increase in leukocyte activation, and a release of inflammatory mediators and growth factors (21, 22). To prevent this problem, some authors have suggested the use of GPIIb/IIIa blockers in combination with platelet activation inhibitors, such as heparin (18, 23), to prevent platelet aggregation and activation. The present invention shows that LB1 was able to simultaneously decrease the activation of GPIIB/IIIa complex, platelet aggregation and externalization of granule surface markers. Platelet granules contain numerous molecules, including coagulation factors, adhesion and cell-activating molecules, cytokines, integrins, inflammatory molecules, and angiogenic factors that play a key role in normal haemostasis, thrombosis and vascular remodelling (24). The striking diminution of granule surface marker externalization in platelets treated with LB1 is an indication that this polypeptide is able to substantially decrease platelet activation. Such a loss platelet expression of surface P-selectin would affect the ability of platelet-leukocyte complexes to form and would alter platelet—endothelial cell adhesion.

### Example 5

### Localization of LB1 Binding on Platelets

### Platelet-Based ELISA

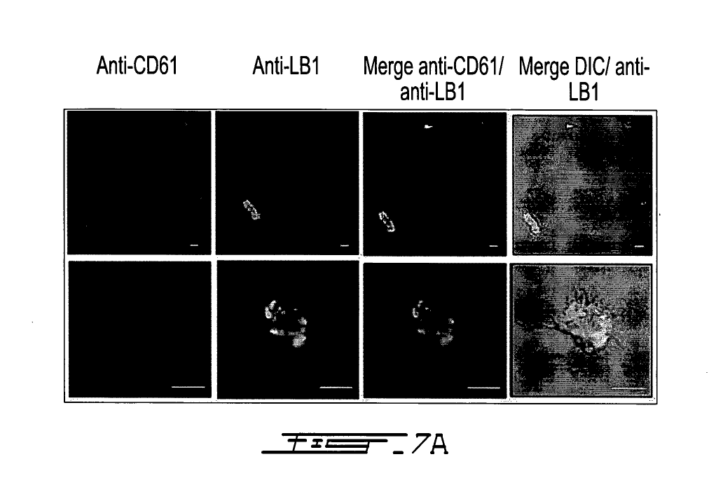

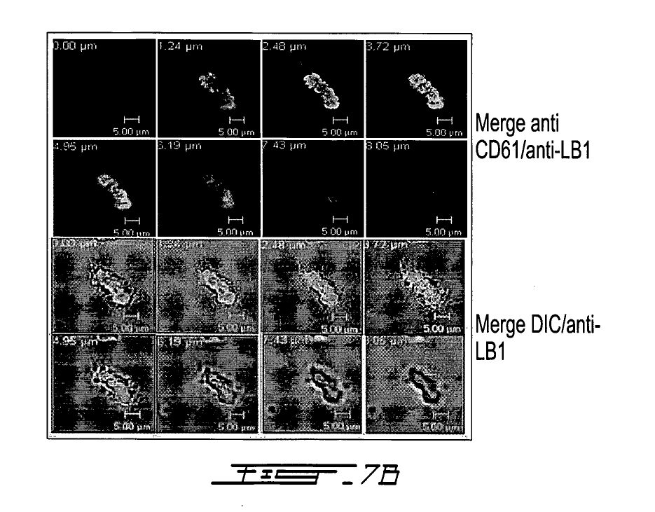

To assess whether the effect of LB1 on platelet activation and aggregation was due to its direct binding to cells, isolated human platelets first activated with 0.05 U/mL of thrombin were exposed to increasing concentrations of LB1 in a platelet-based ELISA. Since platelets are devoid of nuclei, LB1 is considered to be absent from these cells. The absence of LB1 in these cells was confirmed by incubating permeabilized platelets with anti-LB1 antibodies; no binding was detected (data not shown). Therefore, binding of anti-LB1 antibodies requires prior binding of exogenous LB1 to platelets. FIG. 6 shows through OD values, representing the percentage of LB1 binding, that LB1 was able to bind to permeabilized platelets in a dose-dependent manner, and reached a plateau near 200 ng of LB1/10⁶ platelets. Maximum binding corresponded to the active LB1/10⁶ platelets ratio determined in FIGS. 2-4. Binding of NH₂-LB1 to platelets and binding of LB1 to non-permeabilized cells were undetectable (data not shown).

### Indirect Immunofluorescence and Confocal Microscopy

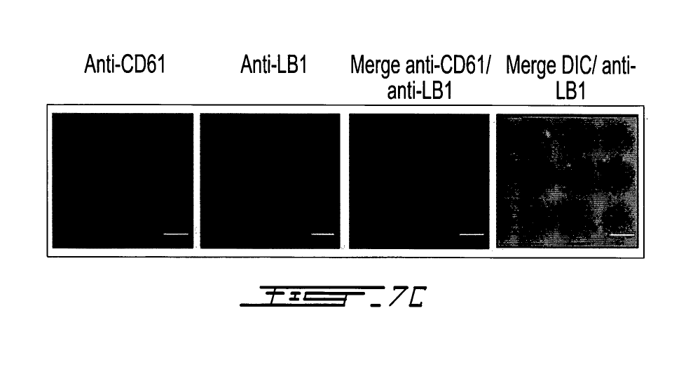

To confirm binding of LB1 to platelets and to localize potential targets, double immunofluorescence experiments as described above were conducted and the results were visualized by confocal laser microscopy. Prior to fixation and permeabilization, non-permeabilized and unfixed platelets were exposed to 200 ng of LB1/10⁶ cells, before activation with 0.05 U/mL thrombin for five minutes and washed to remove any unbound LB1. The LB1 binding pattern was revealed with a mouse anti-LB1 and a FITC-conjugated anti-mouse antibody. Rabbit anti-CD61 and rhodamine-conjugated anti-rabbit antibodies were used as platelet markers. As shown in FIG. 7A, fluorescence due to LB1 binding localized within the activated cells. This finding was confirmed by performing horizontal optical sections of a platelet double-stained for LB1 and CD61 (FIG. 7B). The LB1 staining pattern in these series was entirely compatible with an intracellular distribution. This clearly indicated that LB1 was associated with a continuous structure within the platelets, as evidenced by merging with the digital imaging contrast (DIC) images. However, LB1 was not detected within non-activated platelets (FIG. 7C). The staining pattern of LB1 was not distributed evenly throughout the activated platelets, suggesting a nonuniform distribution of its intracellular target. Indeed, LB1 seemed to form clusters throughout the inside of the platelet.

Approximately 25% of the total platelet population bound LB1. These positive cells displayed multiple unmerged individual filopodia and lamellipodia (FIG. 7B). This suggested that platelets were able to internalize LB1 during a specific temporal window following stimulation. Since all the platelets on a slide were not necessarily activated synchronously, LB1 was probably unable to enter and bind to its intracellular target in all cells.

In order to verify the above hypothesis, anti-CD62 antibodies were used as activation markers. Platelets were exposed to LB1 prior to fixation and permeabilization. As shown in FIG. 7D, LB1 binds to an intracellular target in the activated cells but not in the unactivated ones. Moreover, the polypeptide seemed to enter preferentially within platelets containing P-selectin on the border of the external membrane. The α-granules were present near the external membrane, but the P-selectin was not yet translocated to the surface as shown in the merged DIC/anti-CD62 image (FIG. 7D). These results suggest that internalization of LB1 occurs rapidly during the process of activation, before the externalization of α-granules. Platelets shedding their granules or unactivated platelets are propably unable to bind LB1. Moreover, cells harboring blunt filopodia rotated around the periphery, a morphology characteristic of cells at the end of their activation state, also seemed to stain negative for LB1 binding. (arrow in FIG. 7A).

### Example 6

### Timing of LB1 Effect on Platelets

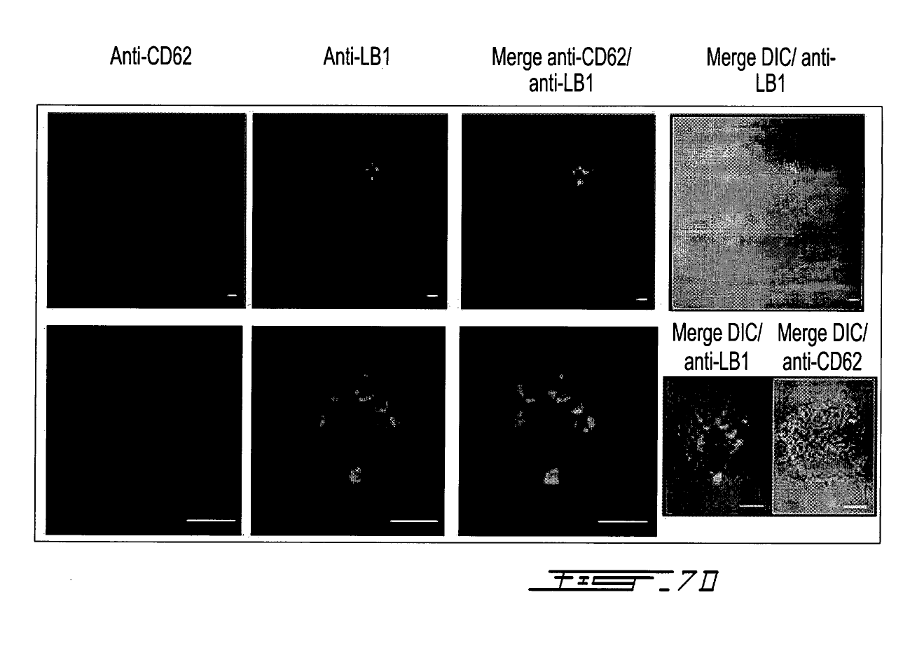

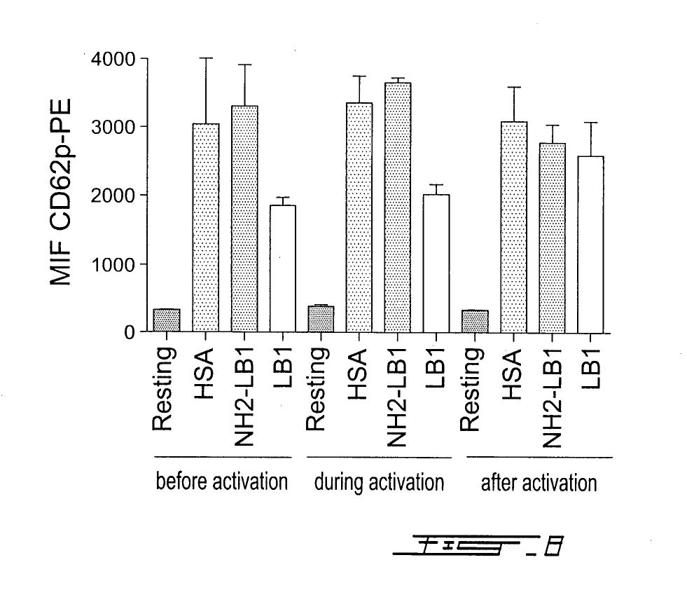

Since LB1 appeared to translocate within platelets only during the process of activation, it was tested whether degranulation of these cells could still occur when LB1 was added after their activation. Isolated human platelets were treated with 200 ng of LB1, HSA or NH₂-LB1/10⁶ cells before, during or after activation with 0.05 U/mL of thrombin. FIG. 8 shows that LB1 did not decrease the. externalization of P-selectin when it was added after platelet activation by thrombin. The mean fluorescence intensity of (MFI) in LB1 treated platelets after activation was 2602±359.6 units compared to 3113.72±355.77 units and 2790.69±188.61 units in cells incubated with HSA and NH₂-LB1, respectively. However, when platelets were incubated with LB1 before and during the activation process, it interfered with the surface expression of P-selectin on the cells. These results suggest that LB1 decreases the externalization of P-selectin during the process of activation.

ELISA and immunofluorescence studies have demonstrated that LB1 binds directly to activated, platelets. The polypeptide appears to bind to an intracellular target that is in close proximity to the external membrane, a finding that implies penetration of the external membrane during the process of activation. However, LB1 is not translocated into all platelets. It seems to bind preferentially to cells at a certain state of activation. Unactivated platelets, typically without any apparent pseudopodia, were negative for the presence of LB1. Activated platelets with unique blunt filopods that extend from the cell centre and are rotated around the cell periphery were also unable to bind LB1. These cells appeared to be at the end of their cycle of structural changes. These results suggest that LB1 is able to enter, bind and exert its inhibitory effects on platelets only during a short period of time. This hypothesis is supported by flow cytometry data. When LB1 was added 15 minutes before or at the time of activation, it was able to successfully diminish P-selectin externalization. However, when it was added after activation, no decrease in α-granule markers externalization was observed. Thus, LB1 seems able to prevent activation but unable to arrest it after it had been initiated. As indicated above, the population of platelets present in thrombosis comprise cells at stages when LB1 can act.

### Example 7

### Effects of LB1 in a Rat Model of Thrombosis

Four rats were treated as described above. In the first animal, no treatment was applied. Complete occlusion was observed at 22 minutes and 18 sec after application of the FeCl₃ solution, which is known to cause occlusion (41). This result is similar to those obtained earlier (41). The thrombus weight was 0.0118 g.

In the second animal, the vehicle, i.e. LB1 buffer only, was injected 5 minutes before application of the FeCl₃ solution. Complete occlusion was observed at 22 minutes and 0 sec after application of the solution. The thrombus weight was 0.0104 g.

In the third animal, 0.6-0.7 mg of LB1 was injected 5 minutes before application of the FeCl₃ solution. Sixty minutes after application of the FeCl₃ solution, there was still a residual blood flow in the artery. The thrombus weight was 0.0053 g.

In the fourth animal, 0.6-0.7 mg of the inactive fragment NH₂-LB1 was injected 5 minutes before application of the FeCl₃ solution. Complete occlusion was observed at 22 minutes and 33 sec after application of the FeCl₃ solution. The thrombus weight was 0.0072 g.

To the Applicant's knowledge, it is the first to show that a nuclear autoantigen can bind to and modulate platelet function. The results presented herein have shown that LB1 is able to significantly suppress platelet activation and aggregation. Furthermore, the results presented herein have demonstrated that LB1 migrates to the inside of platelets during the process of activation and binds to an intracellular target. The present invention indicates that LB1 itself and a C-terminal fragment thereof including the 49 kDa fragment may reduce thrombus formation by inhibiting platelet activation and aggregation, as well as diminish inflammation due to platelet-endothelial cell adhesion by inhibiting the externalization of platelet P-selectin.

Although the present invention has been described hereinabove by way of specific embodiments thereof, it can be modified, without departing from the spirit and nature of the subject invention as defined in the appended claims.

### REFERENCES

1. Shapiro S. The lupus anticoagulant/antiphospholipid syndrom. Annu Rev Med. 1996; 47:533-53.
2. Cines D, McCrae K. The antiphospholipid-protein syndrome. J Clin Immunol. 1995; 15(6 Suppl):86S-100S.
3. Roubey R. Autoantibodies to phospholipid-binding plasma proteins: a new view of lupus anticoagulants and other “antiphospholipid” autoantibodies [see comments]. Blood 1994; 84(9):2854-67.
4. Horbach D, Van Oort E, Donders R, Derksen R, De Groot P. Lupus anticoagulant is the strongest risk factor for both venous and arterial thrombosis in patients with systemic lupus erythematosus. Comparison between different assays for the detection of antiphospholipid antibodies. Thromb Haemost. 1996; 76(6):916-24.
5. Petri M. Epidemiology of the Antiphospholipid Antibody Syndrome. J Autoimmun. 2000; 15(2):145-51.
6. Dieude M, Senecal J, Rauch J, Hanly J, Fortin P, Brassard N, et al. Association of autoantibodies to nuclear lamin B1 with thromboprotection in systemic lupus erythematosus: lack of evidence for a direct role of lamin B1 in apoptotic blebs. Arthritis Rheum. 2002; 46(10):2695-707.
7. Senecal J, Rauch J, Grodzicky T, Raynauld J, Uthman I, Nava A, et al. Strong association of autoantibodies to human nuclear lamin B1 with lupus anticoagulant antibodies in systemic lupus erythematosus. Arthritis Rheum. 1999; 42(7):1347-53.
8. Espinosa G, Cervera R, Font J, Shoenfeld Y. Antiphospholipid syndrome: pathogenic mechanisms. Autoimmun Rev. 2003; 2(2):86-93.
9. Font J, Espinosa G, Tassies D, Pino M, Khamashta M, Gallart T, et al. Effects of beta2-glycoprotein I and monoclonal anticardiolipin antibodies in platelet interaction with subendothelium under flow conditions. Arthritis Rheum. 2002; 46(12):3283-89.
10. Wiener M, Burke M, Fried M, Yust I. Thromboagglutination by Anticardiolipin Antibody Complex in the Antiphospholipid Syndrome: A Possible Mechanism of Immune-Mediated Thrombosis. Thromb Res. 2001; 103(3):193-99.
11. Reverter J C, Tassies D, Font J, Khamashta M, Ichikawa K, Cervera R, et al. Effects of human monoclonal anticardiolipin antibodies on platelet function and on tissue factor expression on monocytes. Arthritis Rheum. 1998; 41(8):1420-27.
12. Shi W, Chong B, Chesterman C. [beta]2-Glycoprotein I is a requirement for anticardiolipin antibodies binding to activated platelets: Differences with lupus anticoagulants. Blood 1993; 81(5):1255-62.
13. Vergnes L, Peterfy M, Bergo M, Young S, Reue K. Lamin B1 is required for mouse development and nuclear integrity. PNAS 2004; 101(28):10428-33.
14. Berden J, Grootscholten C, Jurgen W, van der Vlag J. Lupus nephritis: a nucleosome waste disposal defect? J Nephrol. 2002; 15(Suppl 6):S1-10.
15. Berden J, Grootscholten C, Jurgen W, van der Vlag J. Lupus nephritis:

consequence of disturbed removal of apoptotic cells? Neth J Med. 2003; 61(8):233-38.
16. Dieker J, van der Vlag J, Berden J. Triggers for anti-chromatin autoantibody production in SLE. Lupus. 2002; 11(12):856-64.
17. Lefkovits J, Plow E, Topol E. Platelet Glycoprotein Ilb/Illa Receptors in Cardiovascular Medicine. N Engl J Med 1995; 332(23):1553-59.
18. Day J, Malik I, Weerasinghe A, Poullis M, Nadra I, Haskard D, et al. Distinct yet complementary mechanisms of heparin and glycoprotein IIb/IIIa inhibitors on platelet activation and aggregation: implications for restenosis during percutaneous coronary intervention. Heart 2004; 90(7):794-99.
19. Matzdorff A, Kuhnel G, Kemkes-Matthes B, Voss R. Comparison of GP IIB/IIIA Inhibitors and Their Activity as Measured by Aggregometry, Flow Cytometry, Single Platelet Counting, and the Rapid Platelet Function Analyzer. J Thromb Thrombolysis 2001; 12(2):129-39.
20. Massberg S, Mueller I, Besta F, Thomas P, Gawaz M. Effects of 2 different antiplatelet regimens with abciximab or tirofiban on platelet function in patients undergoing coronary stenting. Am Heart J. 2003; 146(5):E19.
21. lshiwata S, Tukada T, Nakanishi S, Nishiyama S, Seki A. Postangioplasty restenosis: platelet activation and the coagulation-fibrinolysis system as possible factors in the pathogenesis of restenosis. Am Heart J. 1997; 133(4):387-92.
22. Serrano C J, Ramires J, Venturinelli M, Arie S, D'Amico E, Zweier J, et al. Coronary Angioplasty Results in Leukocyte and Platelet Activation With Adhesion Molecule Expression: Evidence of Inflammatory Responses in Coronary Angioplasty. J Am Coll Cardiol. 1997; 29(6):1276-83.
23. Scazziota A, Altman R, Rouvier J, Gonzalez C, Ahmed Z, Jeske W, et al. Abciximab treatment in vitro after aspirin treatment in vivo has additive effects on platelet aggregation, ATP release, and P-selectin expression. Thromb Res. 2000; 100(6):479-88.
24. Reed G. Platelet secretory mechanisms. Semin Thromb Hemost. 2004; 30(4):441-50.
25. Marcu M, Zhang L, Nau-Staudt K, Trifaro J. Recombinant scinderin, an F-actin severing protein, increases calcium-induced release of serotonin from permeabilized platelets, an effect blocked by two scinderin-derived actin-binding peptides and phosphatidylinositol 4,5-bisphosphate. Blood 1996; 87(1):20-24.
26. Flaumenhaft R, Dilks J, Rozenvayn N, Monahan-Earley R, Feng D, Dvorak A. The actin cytoskeleton differentially regulates {alpha}-granule and dense granule secretion. Blood 2005; 105(10):3879-87.
27. Torti M, Festetics E, Bertoni A, Sinigaglia F, Balduini C. Agonist-induced actin polymerization is required for the irreversibility of platelet aggregation. Thromb Haemost. 1996; 76(3):444-49.
28. Hartwig J, Barkalow K, Azim A, Italiano J. The elegant platelet: signals controlling actin assembly. Thromb Haemost 1999; 82(2):392-98.
29. Fox J, Shattil S, Kinlough-Rathbone R, Richardson M, Packham M, Sanan D. The Platelet Cytoskeleton Stabilizes the Interaction between alpha[IMAGE]beta(3) and Its Ligand and Induces Selective Movements of Ligand-occupied Integrin. J. Biol. Chem. 1996; 271(12):7004-11.
30. Schober J, Lam S, Wencel-Drake J. Effect of cellular and receptor activation on the extent of integrin aIIbbIII internalization. J Thromb Haemost 2003; 1(11):2404-10.
31. Shumaker D, Kuczmarski E, Goldman R. The nucleoskeleton: lamins and actin are major players in essential nuclear functions. Curr Opin Cell Biol. 2003; 15(3):358-66.
32. Cohen M, Gruenbaum Y, Lee K, Wilson K. Transcriptional repression, apoptosis, human disease and the functional evolution of the nuclear lamina. Trends Biochem Sci. 2001; 26(1):41-47.
33. Ren Y, Tang J, Mok M, Chan A, Wu A, Lau C. Increased apoptotic neutrophils and macrophages and impaired macrophage phagocytic clearance of apoptotic neutrophils in systemic lupus erythematosus. Arthritis Rheum. 2003; 48(10):2888-97.
34. Herrmann M, Voll R, Zoller O, Hagenhofer M, Ponner B, Kalden J. Impaired phagocytosis of apoptotic cell material by monocyte-derived macrophages from patients with systemic lupus erythematosus. Arthritis Rheum. 1998; 41(7):1241-50.
35. Potter P, Cortes-Hernandez J, Quartier P, Botto M, Walport M. Lupus-prone mice have an abnormal response to thioglycolate and an impaired clearance of apoptotic cells. J Immunol. 2003; 170(6):3223-32.
36. Mevorach D, Zhou J, Song X, Elkon K. Systemic Exposure to Irradiated Apoptotic Cells Induces Autoantibody Production. J. Exp. Med. 1998; 188(2):387-92.
37. Fishelson Z, Attali G, Mevorach D. Complement and apoptosis. Mol Immunol. 2001; 38(2-3):207-19.
38. Cocca B, Cline A, Radic M. Blebs and Apoptotic Bodies Are B Cell Autoantigens. J Immunol 2002; 169(1):159-66.
39. Shoenfeld Y. The idiotypic network in autoimmunity: antibodies that bind antibodies that bind antibodies. Nat Med. 2004; 10(1):17-18.
40. Zinger H, Eilat E, Meshorer A, Mozes E. Peptides based on the complementarity-determining regions of a pathogenic autoantibody mitigate lupus manifestations of (NZB x NZW)F1 mice via active suppression. Int. Immunol. 2003; 15(2):205-14.
41. Lockyer S, and Kambayashi, JI. Demonstration of flow and platelet dependency in a ferric chloride induced model of thrombosis. J. Cardiovasc. Pharmacol. 1999; 33:718-725.

1. A method for preventing a thrombotic event in a patient susceptible to such an event, which comprises the step of administering an effective amount of a lamin B1 nuclear (LB1) antigen to said patient.

2. A method for reducing a thrombotic event in a patient in need for such a treatment, which comprises the step of administering an effective amount of a lamin B1 nuclear (LB1) antigen to said patient.

3. A method as recited in claims 1, wherein the LB1 antigen is a full length LB1.

4. A method as recited in claim 3, wherein the full length LB1 is human.

5. A method as recited in claim 1, wherein the LB1 antigen is a 49 kDa human LB1 C-terminal fragment.

6. A method as recited in claim 1, wherein the effective amount of a LB1 antigen is administered in situ.

7. A method as recited in claim 1, wherein the thrombotic event comprises platelet P-selectin externalization.

8. A method as recited in claim 1, wherein the thrombotic event comprises platelet CD63 externalization.

9. A method as recited in claim 1, wherein the thrombotic event comprises platelet GPIIb/IIIa complex activation.

10. A method as recited in claim 1, wherein the thrombotic event is platelet aggregation.

11. A method as recited in claim 1, wherein the effective amount of a LB1 antigen is administered to the patient prior to platelet activation.

12. A method as recited in claim 1, wherein the effective amount of a LB1 antigen is administered to the patient during platelet activation.

13.-26. (canceled)

27. An anti-thrombotic composition which comprises an effective amount of a lamin B1 nuclear (LB1) antigen and a pharmaceutically acceptable carrier.


SEQUENCE LISTING (1 sequence)

SEQ ID NO: 1 - protein, 586 aa, Homo sapiens
MATATPVPPRMGSRAGGPTTPLSPTRLSRLQEKEELRELNDRLAVYIDKVRSLETENSALQLQVTEREEVRGRELTGLKALYETELADARRALDDTARERAKLQIELGKCKAEHDQLLLNYAKKESDLNGAQIKLREYEAALNSKDAALATALGDKKSLEGDLEDLKDQIAQLEASLAAAKKQLADETLLKVDLENRCQSLTEDLEFRKSMYEEEINETRRKHETRLVEVDSGRQIEYEYKLAQALHEMREQHDAQVRLYKEELEQTYHAKLENARLSSEMNTSTVNSAREELMESRMRIESLSSQLSNLQKESRACLERIQELEDLLAKEKDNSRRMLTDKEREMAEIRDQMQQQLNDYEQLLDVKLALDMEISAYRKLLEGEEERLKLSPSPSSRVTVSRASSSRSVRTTRGKRKRVDVEESEASSSVSISHSASATGNVCIEEIDVDGKFIRLKNTSEQDQPMGGWEMIRKIGDTSVSYKYTSRYVLKAGQTVTIWAANAGVTASPPTDLIWKNQNSWGTGEDVKVILKNSQGEEVAQRSTVFKTTIPEEEEEEEEAAGVVVEEELFHQQGTPRASNRSCAIM